(19) United States
(12) Patent Application Publication
(10) Pub. No.: US 2025/0170376 A1
(43) Pub. Date: May 29, 2025

(54) TRANSFER INSTRUMENT

(71) Applicant: TERUMO KABUSHIKI KAISHA, Tokyo (JP)

(72) Inventor: Koichi Hayakawa, Kanagawa (JP)

(73) Assignee: TERUMO KABUSHIKI KAISHA, Tokyo (JP)

(21) Appl. No.: 18/956,190

(22) Filed: Nov. 22, 2024

(30) Foreign Application Priority Data
Nov. 29, 2023 (JP) 2023-201166

(51) Int. Cl. A61M 31/00

(52) U.S. Cl. CPC A61M 31/00

(57) ABSTRACT

A transfer instrument includes an outer cylinder including a distal-end opening. The outer cylinder includes an outer cylinder main body and a protection portion having a tubular shape, the protection portion including an elastic material and being provided at a distal portion of the outer cylinder main body. A distal portion of the outer cylinder includes a flexible portion that is flexible to undergo curved deformation and is capable of maintaining curved deformation. The protection portion includes an inner covering portion that covers an inner peripheral surface of a most distal end of the outer cylinder main body.

### CROSS-REFERENCES TO RELATED APPLICATIONS

This application claims priority to Japanese Application No. 2023-201166 filed on Nov. 29, 2023, the entire content of which is incorporated herein by reference.

### TECHNOLOGICAL FIELD

The present disclosure relates to a transfer instrument for transferring a medical sheet to a treatment site of a living body.

### BACKGROUND DISCUSSION

Japanese Patent Application Publication No. 2009-000511 A discloses a transfer instrument for transferring a medical sheet (cell sheet) for use in, for example, organ transplantation to a treatment site of a living body. The transfer instrument includes a shaft and a support portion provided at a distal portion of the shaft. The support portion includes a sheet support on which the medical sheet is placed. The transfer instrument is slid while pressing an upper surface of the medical sheet placed on a support surface of the sheet support with forceps or the like to transfer the medical sheet from the support portion to the treatment site.

In the above-described related art, when the medical sheet is transferred from the support surface of the support portion to the treatment site of the living body, it is necessary to manipulate the transfer instrument while pressing the upper surface of the medical sheet with forceps or the like, which raises a concern that the medical sheet cannot be efficiently transferred to the treatment site.

### SUMMARY

(1) An aspect of the present disclosure is a transfer instrument configured to transfer a medical sheet to a treatment site of a living body, the transfer instrument including: an outer cylinder including a distal-end opening; a first carrier member including a first shaft and a support portion having a sheet shape, the first shaft extending in an axial direction of the outer cylinder and being disposed inside the outer cylinder so as to be movable along the axial direction, the support portion being disposed at a distal portion of the first shaft and including a support surface capable of holding the medical sheet; and a second carrier member including a second shaft and a pressure-application portion, the second shaft extending along the first shaft and being provided so as to be movable along the first shaft, the pressure-application portion being provided at a distal portion of the second shaft to press the medical sheet in a distal end direction, in which the outer cylinder includes: an outer cylinder main body having a lumen communicating with the distal-end opening; and a protection portion having a tubular shape, the protection portion including an elastic material and being provided at a distal portion of the outer cylinder main body, at least the distal portion of the outer cylinder main body includes a flexible portion that is flexible to undergo curved deformation and is capable of maintaining curved deformation, the flexible portion is provided at least at a distal portion of the outer cylinder, the protection portion includes an inner covering portion that covers at least an inner peripheral surface of a most distal end of the outer cylinder main body and opens in the distal end direction to communicate with the lumen of the outer cylinder main body, in a state where the first and second shafts are moved in the distal end direction relative to the outer cylinder so as to cause the support portion to protrude from the distal-end opening of the outer cylinder, the support portion is unfolded by being exposed from the outer cylinder in the distal end direction, and in a state where the support portion is unfolded, when the first and second shafts are moved in a proximal end direction relative to the outer cylinder, the support portion is retracted into the lumen of the outer cylinder through the inner covering portion.

This transfer instrument allows the flexible portion to effectively curve the distal portion of the outer cylinder, so that it is possible to efficiently place the support portion of the first carrier member at a desired position. When the support portion is retracted into the lumen of the outer cylinder, the inner covering portion can effectively prevent the support portion from coming into contact with the inner peripheral surface of the most distal end of the flexible portion.

(2) In the transfer instrument according to the above (1), when the support portion is retracted into the lumen of the outer cylinder main body, at least a part of the support portion may be disposed inside the flexible portion.

With this configuration, even in a state where the support portion is retracted in the distal portion of the outer cylinder, the flexible portion can effectively bring the distal portion of the outer cylinder into curved deformation.

(3) In the transfer instrument according to the above (1) or (2), the protection portion may include a protrusion protruding from the most distal end of the outer cylinder main body in the distal end direction.

With this configuration, when the support portion is retracted into the outer cylinder, the protrusion can effectively guide the support portion to the lumen of the outer cylinder.

(4) In the transfer instrument according to the above (3), the protection portion may include an outer covering portion that covers an outer peripheral surface of the flexible portion, and the outer covering portion may extend from the protrusion in the proximal end direction.

With this configuration, when the outer cylinder is inserted into the living body, the outer covering portion can effectively prevent the flexible portion from coming into contact with the living body.

(5) In the transfer instrument according to the above (4), the outer covering portion may cover the outer peripheral surface of the flexible portion over an entire length of the flexible portion in the axial direction of the outer cylinder main body.

With this configuration, when the outer cylinder is inserted into the living body, the outer covering portion can effectively prevent the whole of the flexible portion from coming into contact with the living body.

(6) In the transfer instrument according to the above (1) or (2), the inner covering portion may have a length of half or more of the support portion in the axial direction of the outer cylinder main body.

With this configuration, when the support portion is retracted into the outer cylinder, the outer cylinder main body and the support portion are prevented from coming into direct contact with each other.

(7) In the transfer instrument according to the above (3), the protrusion may be thicker than the inner covering portion in a radial direction of the outer cylinder.

With this configuration, the increase in rigidity of the protrusion allows, when the support portion is retracted into the lumen of the outer cylinder, the protrusion to effectively guide the support portion into the outer cylinder.

(8) In the transfer instrument according to the above (3), an inner peripheral surface of the protrusion may include a reverse tapered portion whose diameter increases toward an end in the distal end direction.

With this configuration, when the support portion is retracted into the lumen of the outer cylinder, the reverse tapered portion can effectively retract the support portion into the outer cylinder by guiding the support portion in the proximal end direction.

(9) In the transfer instrument according to the above (8), the protection portion may include an outer covering portion that covers an outer peripheral surface of the flexible portion and extends from the protrusion in the proximal end direction.

With this configuration, when the outer cylinder is inserted into the living body, the outer covering portion can effectively prevent the flexible portion from coming into contact with the living body.

(10) In the transfer instrument according to the above (1) or (2), the protection portion may include a distal-end covering portion that covers the most distal end of the outer cylinder main body.

With this configuration, the distal-end covering portion can prevent the support portion and the outer cylinder main body from coming into contact with each other more effectively by covering all over the most distal end of the outer cylinder main body.

(11) In the transfer instrument according to the above (1) or (2), the protection portion may include an outer covering portion that covers an outer peripheral surface of the flexible portion, and a distal end of the outer covering portion and a distal end of the inner covering portion may be connected to each other by the distal-end covering portion.

With this configuration, the outer covering portion, the inner covering portion, and the distal-end covering portion can effectively cover the distal portion of the outer cylinder.

(12) In the transfer instrument according to any one of the above (1) to (11), the flexible portion may be a tubular body obtained by winding, in a spiral shape, a plate member including a metal material.

With this configuration, the flexible portion can be made simple and low in cost.

According to the present disclosure, since at least the distal portion of the outer cylinder main body includes the flexible portion that is flexible to undergo curved deformation and is capable of maintaining curved deformation, it is possible to efficiently place the support portion of the first carrier member at a desired position by effectively curving the distal portion of the outer cylinder. The cylindrical protection portion including an elastic material is provided at the distal portion of the outer cylinder main body, and the inner peripheral surface of the most distal end of the outer cylinder main body is covered with the inner covering portion. Therefore, when the support portion is retracted into the lumen of the outer cylinder, it is possible to effectively prevent the support portion from coming into contact with the inner peripheral surface of the most distal end of the flexible portion.

(13) A transfer instrument configured to transfer a medical sheet to a treatment site of a living body is disclosed, the transfer instrument comprising: an outer cylinder including a distal-end opening; a first carrier member including a first shaft and a support portion having a sheet shape, the first shaft extending in an axial direction of the outer cylinder and being disposed inside the outer cylinder so as to be movable along the axial direction, the support portion being disposed at a distal portion of the first shaft and including a support surface configured to hold the medical sheet; a second carrier member including a second shaft and a pressure-application portion, the second shaft extending along the first shaft and being provided so as to be movable along the first shaft, the pressure-application portion being provided at a distal portion of the second shaft to press the medical sheet in a distal end direction; the outer cylinder including an outer cylinder main body having a lumen communicating with the distal-end opening and a protection portion having a tubular shape, the protection portion including an elastic material and being provided at a distal portion of the outer cylinder main body; at least the distal portion of the outer cylinder main body includes a flexible portion that is flexible to undergo curved deformation and is configured to maintain a curved deformation; the flexible portion is provided at least at a distal portion of the outer cylinder; the protection portion includes an inner covering portion that covers at least an inner peripheral surface of a most distal end of the outer cylinder main body and opens in the distal end direction to communicate with the lumen of the outer cylinder main body; and wherein the protection portion includes a protrusion protruding from the most distal end of the outer cylinder main body in the distal end direction, and an outer covering portion that covers an outer peripheral surface of the flexible portion, the outer covering portion extending from the protrusion in the proximal end direction, and the outer covering portion covers the outer peripheral surface of the flexible portion over an entire length of the flexible portion in the axial direction of the outer cylinder main body.

(14) A method is disclosed for transferring a medical sheet to a treatment site of a living body, the method comprising: placing a medical sheet on a support surface of a medical instrument, the medical instrument including an outer cylinder including a distal-end opening, a first carrier member including a first shaft and a support portion having a sheet shape, the first shaft extending in an axial direction of the outer cylinder and being disposed inside the outer cylinder so as to be movable along the axial direction, the support portion being disposed at a distal portion of the first shaft and the support surface; and pressing the medical sheet in a distal end direction with a second carrier member, the second carrier member including a second shaft and a pressure-application portion, the second shaft extending along the first shaft and being provided so as to be movable along the first shaft, the pressure-application portion being provided at a distal portion of the second shaft, and wherein the outer cylinder includes an outer cylinder main body having a lumen communicating with the distal-end opening and a protection portion having a tubular shape, the protection portion including an elastic material and being provided at a distal portion of the outer cylinder main body.

### BRIEF DESCRIPTION OF THE DRAWINGS

FIG. 1 is a perspective view of a transfer instrument according to an embodiment of the present disclosure.

FIG. 2 is an exploded perspective view of the transfer instrument illustrated in FIG. 1.

FIG. 3 is a plan view of a distal portion of the transfer instrument illustrated in FIG. 1.

FIG. 4 is a longitudinal cross-sectional view taken along line IV-IV in FIG. 3.

FIG. 5 is a cross-sectional view of a configuration where a proximal end of a first support portion is held by a carrier holding portion and a tube.

FIG. 6 is a transverse cross-sectional view taken along line VI-VI in FIG. 3.

FIG. 7A is an enlarged cross-sectional view of a distal portion of an outer cylinder illustrated in FIG. 4.

FIG. 7B is an enlarged cross-sectional view of a configuration where a protection portion enters a step portion of a flexible portion.

FIG. 8 is a flowchart illustrating a procedure of a transfer method for transferring a medical sheet using the transfer instrument illustrated in FIG. 1.

FIG. 9 is a first explanatory diagram of a sheet placing process.

FIG. 10 is a second explanatory diagram of the sheet placing process.

FIG. 11 is a first explanatory diagram of a retracting process.

FIG. 12 is a second explanatory diagram of the retracting process.

FIG. 13 is a longitudinal cross-sectional view taken along line XIII-XIII in FIG. 12.

FIG. 14 is a transverse cross-sectional view taken along line XIV-XIV in FIG. 12.

FIG. 15 is a transverse cross-sectional view taken along line XV-XV in FIG. 12.

FIG. 16 is an explanatory diagram of a positioning process.

FIG. 17 is an explanatory diagram of an unfolding process.

FIG. 18 is an explanatory diagram of a moving process.

FIG. 19 is an explanatory diagram of a withdrawing process.

FIG. 20 is an enlarged cross-sectional view of a distal portion of a transfer instrument according to a first modification.

FIG. 21A is an enlarged cross-sectional view of a distal portion of an outer cylinder of the transfer instrument illustrated in FIG. 20.

FIG. 21B is an enlarged cross-sectional view of a configuration where a protection portion enters a step portion of a flexible portion.

FIG. 22 is an enlarged cross-sectional view of a distal portion of a transfer instrument according to a second modification.

FIG. 23A is an enlarged cross-sectional view of a distal portion of an outer cylinder of the transfer instrument illustrated in FIG. 22.

FIG. 23B is an enlarged cross-sectional view of a configuration where a protection portion enters a step portion of a flexible portion.

FIG. 24 is an enlarged cross-sectional view of a distal portion of a transfer instrument according to a third modification.

FIG. 25A is an enlarged cross-sectional view of a distal portion of an outer cylinder of the transfer instrument illustrated in FIG. 24.

FIG. 25B is an enlarged cross-sectional view of a configuration where a protection portion enters a step portion of a flexible portion.

### DETAILED DESCRIPTION

Set forth below with reference to the accompanying drawings is a detailed description of embodiments of a transfer instrument.

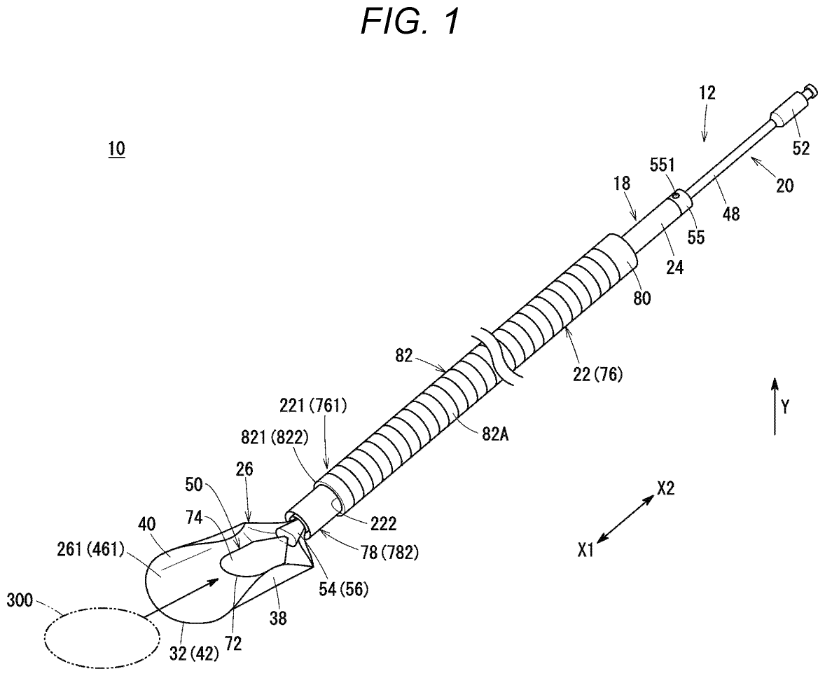

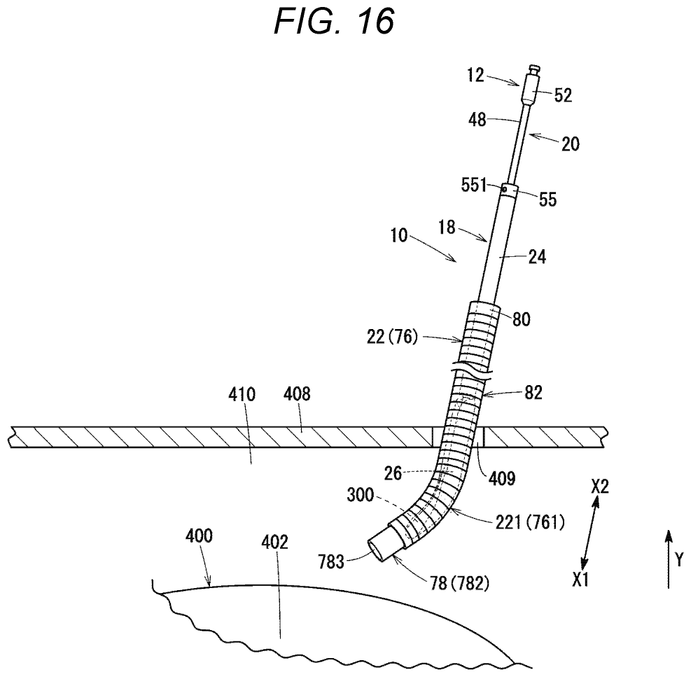

As illustrated in FIG. 1, a transfer instrument 10 according to the present embodiment is a medical instrument for transferring a medical sheet 300 to a treatment site of a living body. The transfer instrument 10 can be used for, for example, the treatment of severe heart failure caused by ischemic heart disease. In this case, the medical sheet 300 is transplanted to a recipient site 402 of a heart 400 (the treatment site of the living body) (see FIGS. 16 to 19). The transfer instrument 10 is capable of attaching a plurality of the medical sheets 300 to the recipient site 402.

Examples of such a medical sheet 300 include pharmaceutical products or regenerative medicine products for medical use, a medical instrument, and the like. The medical sheet 300 is formed in a sheet shape such as a film shape or a membrane shape (gel object). Fibrin or the like may be applied to the medical sheet 300 for reinforcement. Examples of the regenerative medicine products including cells include a cell sheet (sheet-shaped cell culture), a spheroid, and the like. It is possible to form the cell sheet by culturing autologous cells or allogenic cells. The cells constituting the cell sheet can include, for example, somatic stem cells (adult stem cells), mesenchymal stem cells, or iPS cells (induced pluripotent stem cells)-derived cardiomyocytes. Examples of the somatic stem cells preferably include skeletal myoblast cells (myoblast cells).

The medical sheet 300 may contain a tissue adhesive, a local anesthetic, or the like. The medical sheet 300 can have a thickness of, for example, about 100 μm, and can have a diameter of, for example, about 40 mm. Note that the thickness and the diameter (size) of the medical sheet 300 can be set as desired.

The medical sheet 300 may be a sheet to be transplanted to an organ (for example, lung, liver, pancreas, kidney, small intestine, esophagus, or the like) other than the heart 400. Further, the medical sheet 300 may be, for example, an anti-adhesion sheet as long as the sheet is for medical use.

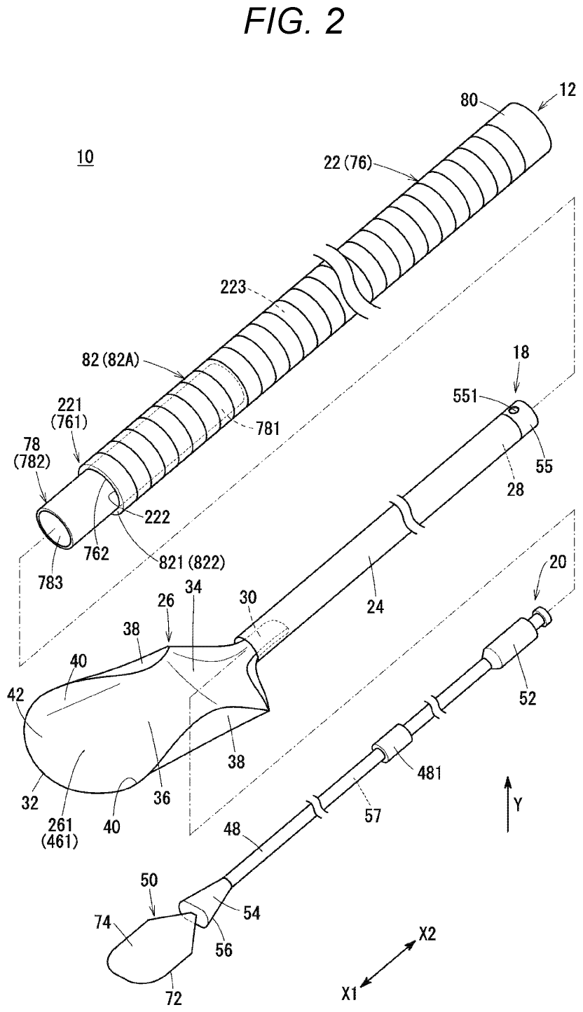

As illustrated in FIG. 1, the transfer instrument 10 includes an instrument body 12. The instrument body 12 includes a first carrier member 18, a second carrier member 20, and an outer cylinder 22. As illustrated in FIG. 2, the first carrier member 18 includes a first shaft 24 and a first support portion 26.

The first shaft 24 is a tubular body (in the present embodiment, a circular tube member) having a first lumen 28. The first lumen 28 opens at a distal end (end in a direction of arrow X1) of the first shaft 24 and opens at a proximal end (end in a direction of arrow X2) of the first shaft 24. The first shaft 24 is provided with, at its proximal end, an airtight valve 55 that is in close contact with an outer peripheral surface of a second shaft 48. The valve 55 is provided with a marker 551 on its outer peripheral surface. When the transfer instrument 10 is used, the marker 551 is visible to the user. Note that the first shaft 24 is not limited to a tubular body and may be a body other than a tubular body.

The first shaft 24 extends in an axial direction of the outer cylinder 22 and is disposed inside the outer cylinder 22 so as to be movable along the axial direction. The first shaft 24 can include, for example, a resin material. Examples of the constituent material of the first shaft 24 include, but not particularly limited to, polyethylene, polypropylene, fluororesin, polyethylene terephthalate, polymethyl methacrylate, a polyamide resin, polystyrene, polycarbonate, polyimide, polyetherimide, polyetheretherketone, polyvinyl chloride, an ABS resin, a polyamide elastomer, and a polyester elastomer. The first shaft 24 may include a metal material.

The first shaft 24 may be flexible. The first shaft 24 may have a flexible tube portion capable of maintaining a bent shape. In this case, the first shaft 24 can be bent into an any desired shape in a body cavity and is capable of maintaining the bent shape.

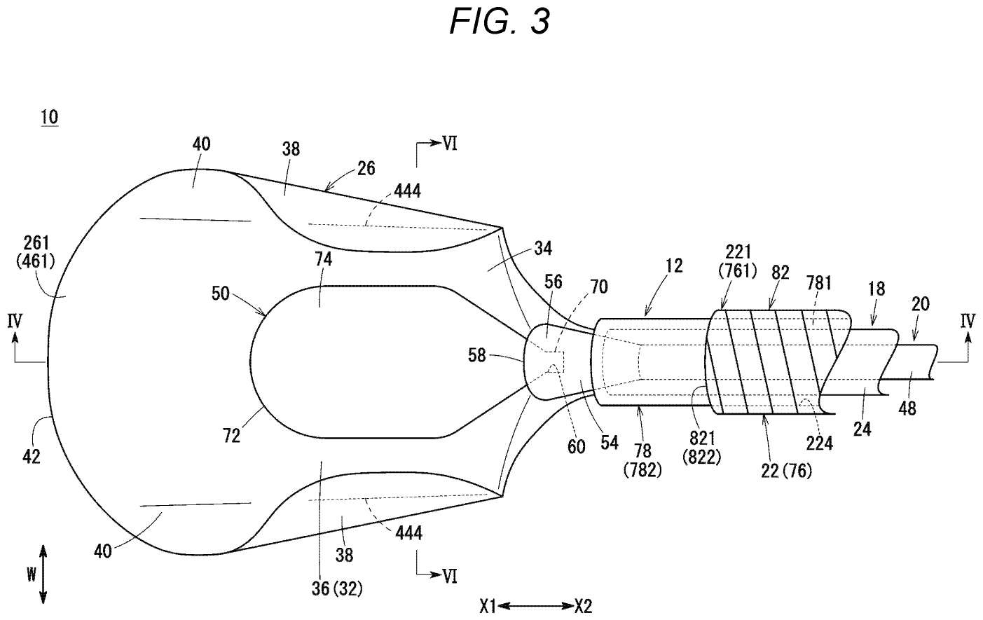

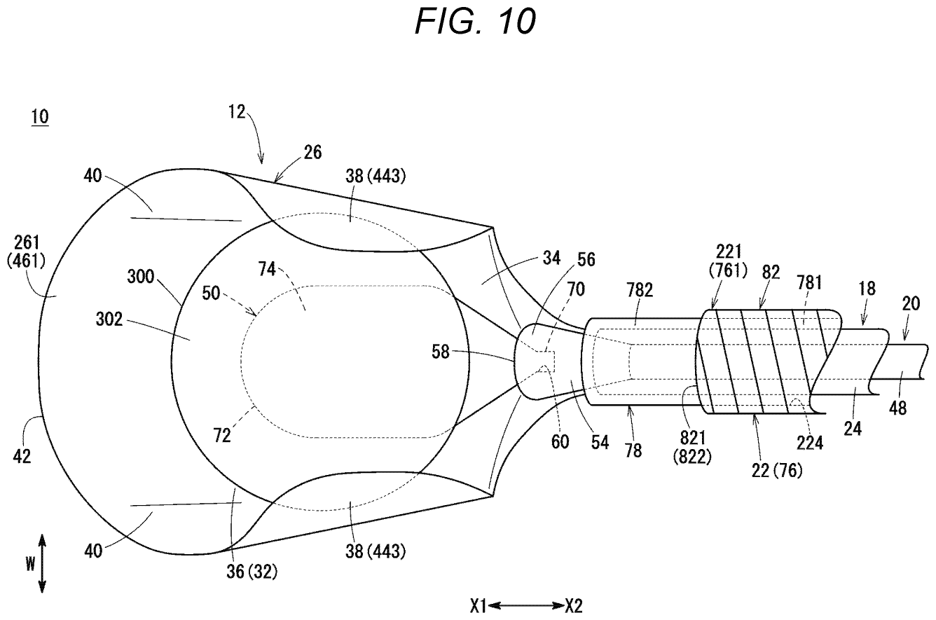

As illustrated in FIG. 3, the first support portion 26 is attached to a distal portion of the first shaft 24. The first support portion 26 can include, for example, a resin material. The first support portion 26 can hold the medical sheet 300 (see FIG. 10). A flexible resin sheet member (film member) is bent into a predetermined form to form the first support portion 26. Alternatively, the sheet member is shaped into a predetermined form by a sheet forming die to form the first support portion 26. It is preferable that the sheet member have, but not particularly limited to, a thickness of, for example, 100 μm or more and 200 μm or less. The first support portion 26 includes a first joint 30 and a first support body 32.

The constituent material of the first support portion 26 preferably has transparency, and examples of the constituent material can include, but not particularly limited to, polyethylene, polycarbonate, polyamide, polystyrene, polypropylene, a polyacetal resin, polyimide, polyetherimide, polyetheretherketone, polyethylene terephthalate, and fluororesin. Further, the first support portion 26 may have a mesh shape.

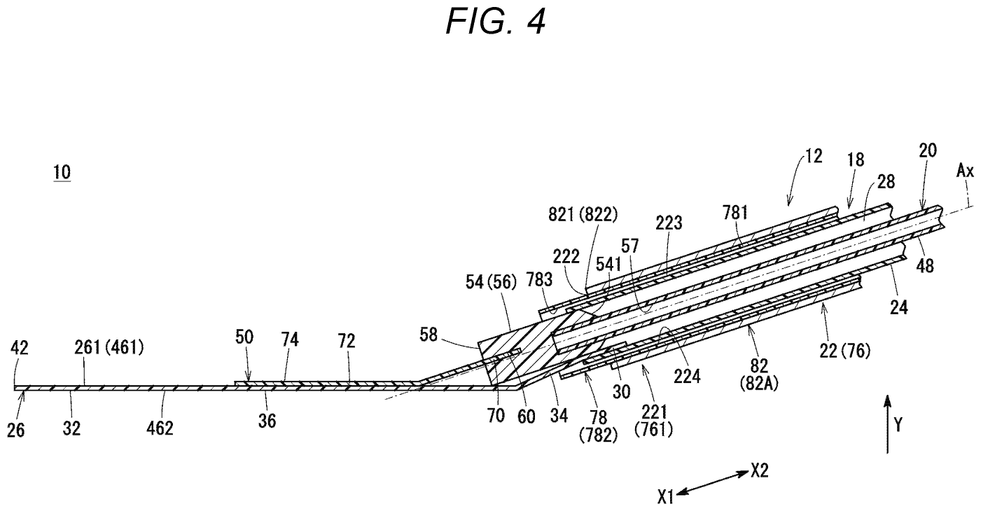

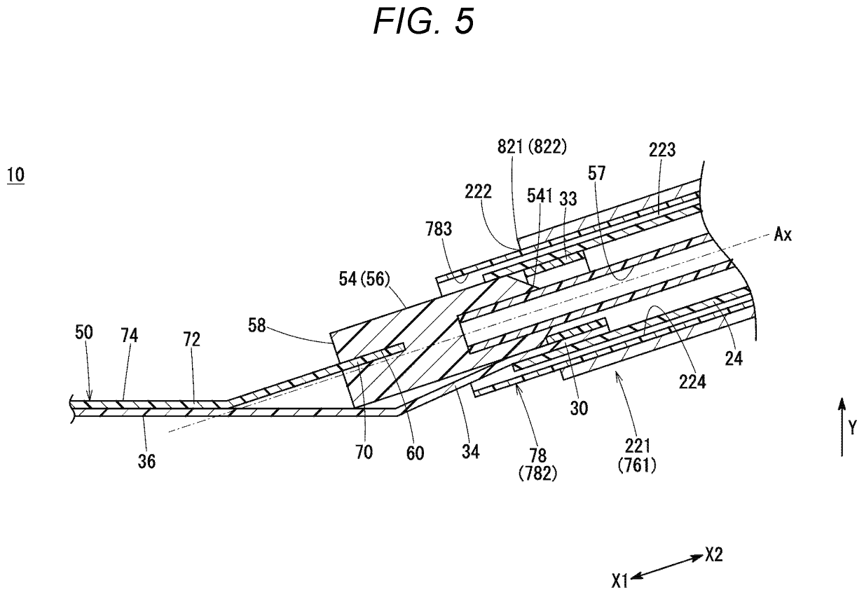

In FIG. 4, the first joint 30 is bonded to an inner peripheral surface of the distal portion of the first shaft 24 with an adhesive. Examples of the adhesive can include, but not particularly limited to, a UV adhesive, a hot-melt adhesive, and an instant adhesive (for example, cyanoacrylate-based instant adhesive). The first joint 30 may be thermally fused to the inner peripheral surface of the first shaft 24. Note that, as illustrated in FIG. 5, a tube 33 smaller in diameter than the first shaft 24 may be disposed in the first lumen 28 of the first shaft 24, and a proximal portion of the first joint 30 may be bonded and fixed to the first shaft 24 with being inserted and sandwiched between the first shaft 24 and the tube 33. In this case, the tube 33 covers up to a proximal end of the first joint 30 in the axial direction of the first shaft 24. A distal end of the tube 33 is disposed adjacent to the proximal end (in the direction of arrow X2) relative to a distal end of the first joint 30. Further, the first support portion 26 may be detachable from the distal portion of the first shaft 24.

As illustrated in FIG. 2, the first support body 32 extends in a distal end direction from the first joint 30. The first support body 32 includes a proximal-end support portion 34, an intermediate support portion 36, a pair of first protrusions 38, a pair of second protrusions 40, and a distal-end support portion 42. The first support body 32 includes a first support surface 261. The marker 551 of the first shaft 24 is disposed so as to face upward when the first support surface 261 faces upward. That is, the marker 551 allows the user to confirm the direction of the first support surface 261 of the first support portion 26.

As illustrated in FIG. 3, the proximal-end support portion 34 is formed to be wider in its extending direction. In other words, both sides of the proximal-end support portion 34 in a width direction are tapered toward the first joint 30. The intermediate support portion 36 is formed in a tapered shape so as to be gradually narrower in width from the distal end toward the proximal end (in the direction of arrow X2).

The distal-end support portion 42 is connected to a distal end of the intermediate support portion 36 and distal ends of the pair of second protrusions 40. The distal-end support portion 42 protrudes in an arc shape in the distal end direction (the direction of arrow X1). That is, when viewed from a direction perpendicular to the first support surface 261 illustrated in FIG. 3, the distal-end support portion 42 of the first support portion 26 has an arc shape connecting the pair of second protrusions 40.

As illustrated in FIG. 4, the proximal-end support portion 34 extends roughly along an axis Ax of the first shaft 24 from the distal end of the first joint 30 in the distal end direction (the direction of arrow X1). The intermediate support portion 36 intersects with the axis Ax of the first shaft 24 and extends from a distal end of the proximal-end support portion 34 toward a distal end of the first support portion 26 (in the direction of arrow X1).

As illustrated in FIG. 3, the pair of first protrusions 38 protrude upward (in a direction of arrow Y) from both sides of the intermediate support portion 36 in the width direction orthogonal to a direction in which the first shaft 24 moves and inward in the width direction of the intermediate support portion 36. The pair of first protrusions 38 are connected to the proximal-end support portion 34. Each first protrusion 38 has a convex shape extending in a direction away from the first support surface 261.

The pair of second protrusions 40 are connected to distal ends of the pair of first protrusions 38. The pair of second protrusions 40 protrude upward from both sides of the intermediate support portion 36 in the width direction (in a direction of arrow W) and outward in the width direction of the intermediate support portion 36. The second protrusions 40 are formed with a smaller curvature than the first protrusions 38. The second protrusions 40 are lower in protrusion height relative to the first support surface 261 than the first protrusions 38 (see FIG. 2).

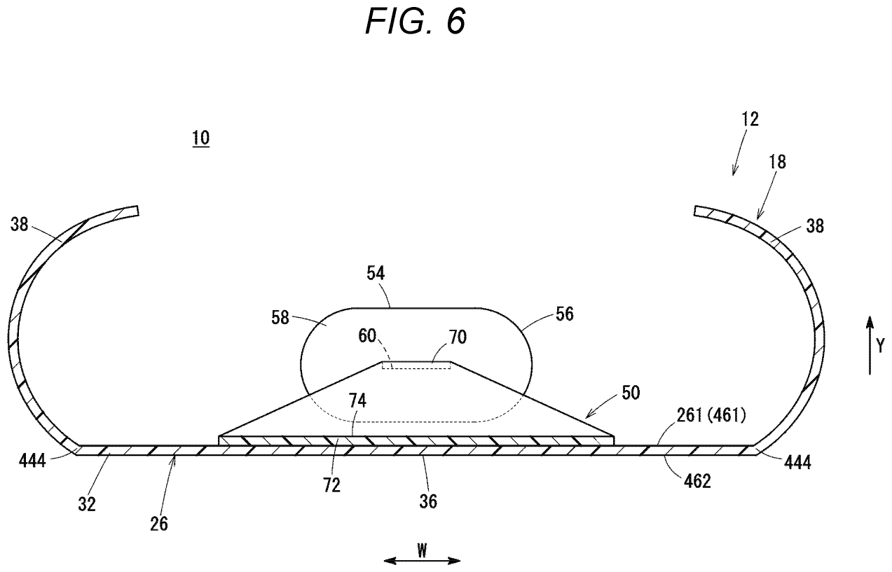

Each of the pair of first protrusions 38 has one of a pair of bent portions 444. Each of the pair of bent portions 444 causes a corresponding one of the pair of first protrusions 38 to bend from the first support surface 261 (intermediate support portion 36) of the first support portion 26 (see FIG. 6).

The first support body 32 includes a front surface 461 that faces upward (in the direction of arrow Y) and includes the first support surface 261, and a back surface 462 that is a surface opposite to the front surface 461. The first support surface 261 includes a flat surface continuous over an upper surface of the proximal-end support portion 34 and upper surfaces of the intermediate support portion 36 and the distal-end support portion 42. A lubricant may be applied to the first support surface 261 so as to allow a second support portion 50 (to be described later) of the second carrier member 20 to smoothly slide on the first support surface 261.

As illustrated in FIG. 2, the second carrier member 20 includes the second shaft 48, the second support portion 50, and a hub 52.

The second shaft 48 is a tubular body (in the present embodiment, a circular tube member) having a second lumen 57. The second shaft 48 is longer in the axial direction than the first shaft 24. The second shaft 48 is inserted into the first lumen 28 of the first shaft 24 (see FIG. 4). In other words, a distal portion of the second shaft 48 protrudes in the distal end direction (the direction of arrow X1) from the distal-end opening of the first shaft 24. A proximal portion of the second shaft 48 protrudes in the proximal end direction (the direction of arrow X2) from the proximal-end opening of the first shaft 24 (see FIG. 1). The second shaft 48 is provided extending along the first shaft 24 so as to be movable along the first shaft 24. Note that the second shaft 48 is not limited to a tubular body and may be a body other than a tubular body.

The second shaft 48 is configured to follow the shape of the first support portion 26. As the constituent material of the second shaft 48, for example, a material more flexible than the constituent material of the first shaft 24 is selected. Specifically, examples of the constituent material of the second shaft 48 can include a polyamide elastomer, a polyester elastomer, a polyurethane elastomer, polyvinyl chloride, polybutadiene, a silicone rubber, and a metal coil (including a composite with a resin). The second shaft 48 is flexible.

As illustrated in FIG. 4, the second shaft 48 includes a carrier holding portion 54 and a pressure-application portion 56 that is the distal portion of the second shaft 48. The pressure-application portion 56 includes an elastic body such as an elastomer member. The pressure-application portion 56 presses the first support portion 26 against an inner surface of the outer cylinder 22 with the first support portion 26 retracted in the outer cylinder 22.

A distal end of the carrier holding portion 54 includes a pressing surface 58. The carrier holding portion 54 can cause the pressing surface 58 to press an outer edge surface of the medical sheet 300 supported by the first support portion 26 in the distal end direction (the direction of arrow X1). In the present embodiment, the pressure-application portion 56 is provided with the carrier holding portion 54 that supports the second support portion 50. The carrier holding portion 54 includes the pressing surface 58 and an attachment hole 60.

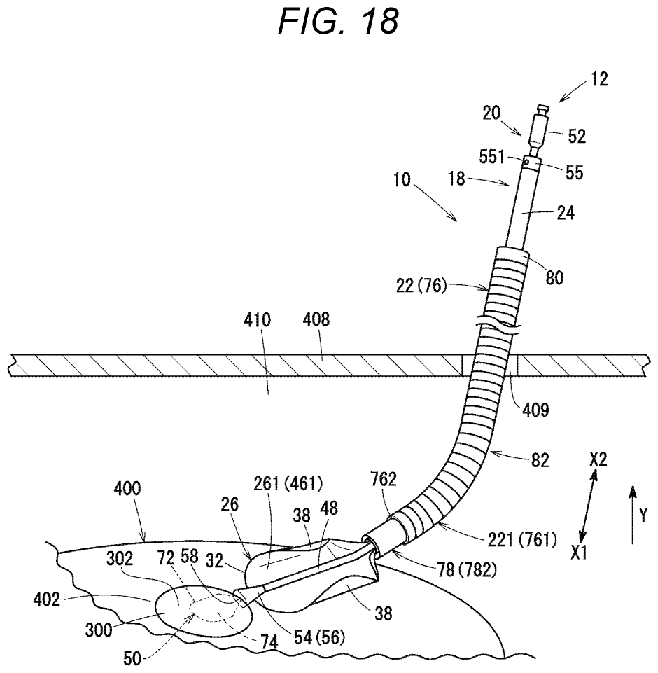

In FIG. 6, the pressing surface 58 is provided on a distal end surface of the carrier holding portion 54. The attachment hole 60 opens at the pressing surface 58. The second support portion 50 is attached to the pressing surface 58. The pressing surface 58 presses the outer edge surface of the medical sheet 300 in the distal end direction (the direction of arrow X1) (see FIG. 18). As illustrated in FIG. 4, a proximal end of the carrier holding portion 54 includes an insertion portion 541 tapered in the proximal end direction. When the insertion portion 541 is inserted into the first lumen 28 from the distal end of the first shaft 24, the carrier holding portion 54 is held at the distal end of the first shaft 24. The insertion portion 541 allows the proximal end of the carrier holding portion 54 to be easily inserted into the first lumen 28 from the distal end of the first shaft 24.

As illustrated in FIG. 3, the second support portion 50 is a flexible sheet. The second support portion 50 includes a second joint 70 and a second support body 72. The second joint 70 is provided at a proximal end of the second support portion 50. The second joint 70 is provided at a proximal end of the second support body 72. The second joint 70 is inserted into and, for example, bonded to the attachment hole 60 of the carrier holding portion 54. The second joint 70 may be joined to the attachment hole 60 of the carrier holding portion 54 by a suitable joining method other than adhesion. Further, the second support portion 50 may be integrally formed with the carrier holding portion 54.

The second support body 72 extends in the distal end direction (the direction of arrow X1) from the second joint 70. The second support body 72 extending from the second joint 70 is shorter in the extending direction than the first support body 32 extending from the first joint 30. The second support body 72 is provided with, on its upper surface, a second support surface 74 on which the medical sheet 300 is placed. The second support surface 74 includes a flat surface. The second support body 72 is smaller than the first support body 32. That is, the second support surface 74 is smaller in area than the first support surface 261.

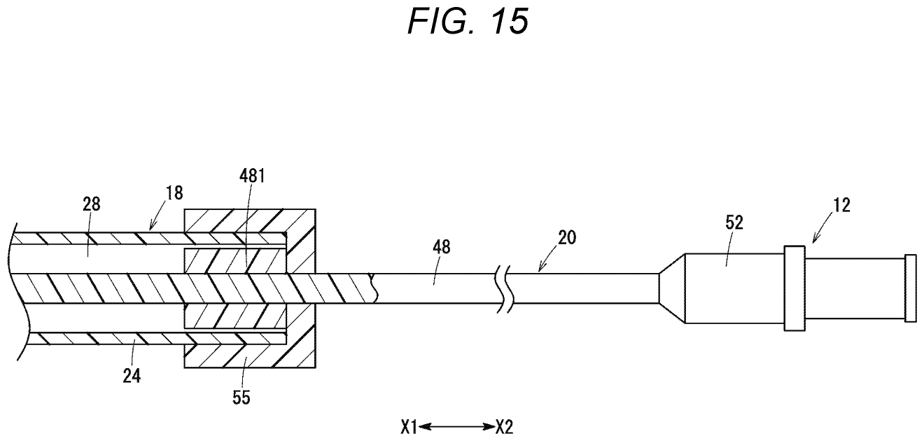

In FIG. 2, the hub 52 is attached to the proximal portion of the second shaft 48. The second carrier member 20 further includes a stopper 481. The stopper 481 is disposed away from the hub 52 in the distal end direction (the direction of arrow X1). The stopper 481 is larger in diameter than the second shaft 48. The stopper 481 is inserted into the first lumen 28 of the first shaft 24 (see FIG. 15). When the second shaft 48 is pulled in the proximal end direction relative to the first shaft 24, the stopper 481 comes into contact with the inside of the valve 55 to be caught (see FIG. 15). When the operation of pulling the second shaft 48 in the proximal end direction is performed, the stopper 481 allows, without applying an excessive load to the proximal portion of the carrier holding portion 54, the first support body 32 to be pulled and the first support portion 26 to be retracted into the outer cylinder 22.

The outer cylinder 22 is a cylindrical member having a distal-end opening 222 and a lumen 223 communicating with the distal-end opening 222. The outer cylinder 22 includes an outer cylinder main body 76 having the lumen 223 and a cylindrical protection portion 78 that includes an elastic material and is provided at a distal portion 761 of the outer cylinder main body 76. The lumen 223 has the distal-end opening 222 that opens at a distal end (end in the direction of arrow X1) of the outer cylinder main body 76. The lumen 223 opens at a proximal end (end in the direction of arrow X2) of the outer cylinder main body 76.

The first shaft 24 is inserted into the lumen 223 of the outer cylinder 22 (see FIG. 4). The outer cylinder 22 is shorter in the axial direction than the first shaft 24. The outer cylinder 22 is provided with, at its proximal end, an airtight valve 80 that is in close contact with an outer peripheral surface of the first shaft 24.

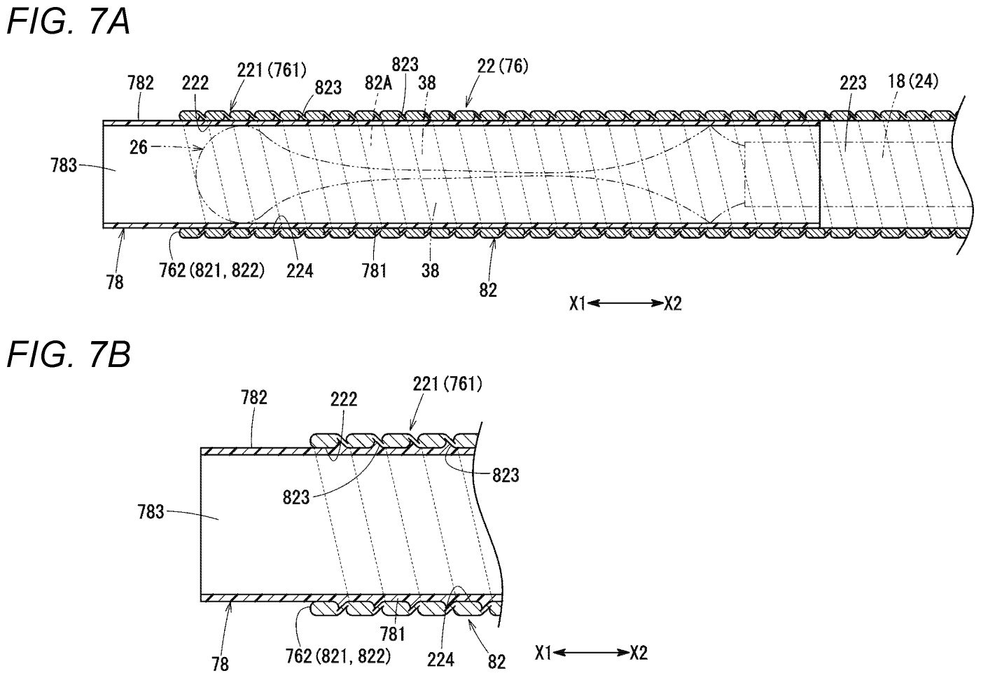

The outer cylinder main body 76 includes a flexible portion 82 that is flexible to undergo curved deformation and is capable of maintaining curved deformation. The flexible portion 82 is a tubular body obtained by winding, in a spiral shape, plate members 82A including a metal material. The flexible portion 82 is provided all over the outer cylinder main body 76 in the axial direction from the distal portion 761 to the proximal portion of the outer cylinder main body 76. That is, the outer cylinder 22 is a flexible tube including the flexible portion 82. As illustrated in FIG. 7A, for example, the flexible portion 82 has the plate members 82A spirally arranged adjacent to each other in the axial direction of the flexible portion 82, and has a step portion 823 recessed in a radial direction provided between the adjacent plate members 82A. A most distal end 821 of the flexible portion 82 coincides with a most distal end 762 of the outer cylinder main body 76. Note that the flexible portion 82 is not limited to the configuration where the flexible portion 82 is provided all over the outer cylinder main body 76 in the axial direction. The flexible portion 82 may be provided at least at the distal portion of the outer cylinder main body 76 and at least at the distal portion of the outer cylinder 22. For example, the flexible portion 82 may have a length of ½ of the entire length of the outer cylinder main body 76 from the distal portion 761 of the outer cylinder main body 76 (the most distal end 821), or may have a length of ⅓ of the entire length of the outer cylinder main body 76 from the distal portion 761 of the outer cylinder main body 76.

In the flexible portion 82, the plate members 82A adjacent each other in the extending direction of the outer cylinder main body 76 are engaged so as to be able to approach each other or separate from each other. The pitch of the plate members 82A of the flexible portion 82 in the axial direction is uniform in the axial direction. Note that the flexible portion 82 is not limited to a tubular body obtained by winding, in a spiral shape, the plate members 82A including a metal material. The flexible portion 82 may include, for example, a resin material as long as it is flexible to undergo curved deformation and is capable of maintaining curved deformation. The pitch of the plate members 82A of the flexible portion 82 is not limited to the uniform pitch in the axial direction. For example, the pitch of the plate members 82A may be smaller at the distal portion of the flexible portion 82.

The protection portion 78 includes an inner covering portion 781 to be inserted into the lumen 223 of the outer cylinder main body 76 and a protrusion 782 protruding in the distal end direction from the most distal end 762 of the outer cylinder main body 76. The protection portion 78 is formed in a tubular shape extending in the axial direction. The protection portion 78 can include, for example, an elastic resin material. For example, examples of the constituent material of the protection portion 78 include, but not particularly limited to, a soft material such as fluororesin, polyethylene, a polyamide elastomer, polyurethane, a polyester elastomer, and a polystyrene elastomer. The protection portion 78 can include, for example, a heat-shrinkable tube that is shrinkable by heating. A through hole 783 is formed inside the protection portion 78 along the axial direction of the protection portion 78. When the protection portion 78 is provided at the distal portion 761 of the outer cylinder main body 76, the through hole 783 and the lumen 223 of the outer cylinder main body 76 communicate with each other.

The inner covering portion 781 is provided at the distal portion 761 of the outer cylinder main body 76. The inner covering portion 781 covers all around an inner peripheral surface 224 of at least the most distal end of the outer cylinder main body 76 (flexible portion 82). Note that the inner covering portion 781 is not limited to the configuration where the inner covering portion 781 is provided only at the distal portion 761 of the outer cylinder main body 76. For example, the inner covering portion 781 may be disposed so as to cover all around the inner peripheral surface 224 of the outer cylinder main body 76 in the extending direction of the outer cylinder main body 76. The inner covering portion 781 covers the step portion 823 of the flexible portion 82. A space is provided between the step portion 823 and the inner covering portion 781. Note that the inner covering portion 781 is not limited to the configuration where the inner covering portion 781 covers the inner peripheral surface 224 of the flexible portion 82 with a space provided between the inner covering portion 781 and the step portion 823. For example, as illustrated in FIG. 7B, a part of the inner covering portion 781 may be configured to enter the space formed by the step portion 823. The part of the inner covering portion 781 can enter the space formed by the step portion 823 by, for example, fusion or insert molding. In this configuration, when the flexible portion 82 undergoes curved deformation, the part of the inner covering portion 781 entering the step portion 823 may be pushed radially inward.

A length of the inner covering portion 781 in the extending direction of the protection portion 78 is longer than or equal to a length of the first support portion 26 with the first support portion 26 retracted in the lumen 223 of the outer cylinder 22. Note that the inner covering portion 781 is not limited to the configuration where the inner covering portion 781 is longer than or equal to a length of the first support portion 26. For example, the length of the inner covering portion 781 may be shorter than the entire length of the first support portion 26. For example, the length of the inner covering portion 781 may be half or more of the entire length of the first support portion 26.

The protrusion 782 is provided at a distal end of the inner covering portion 781. The protrusion 782 extends in the distal end direction (the direction of arrow X1) relative to the inner covering portion 781. In the radial direction of the protection portion 78, the protrusion 782 and the inner covering portion 781 are almost identical in thickness. The protrusion 782 opens in the distal end direction. The through hole 783 of the protrusion 782 and the lumen 223 of the outer cylinder 22 communicate with each other through the inner covering portion 781. Note that the protection portion 78 may include only the inner covering portion 781 without including the protrusion 782.

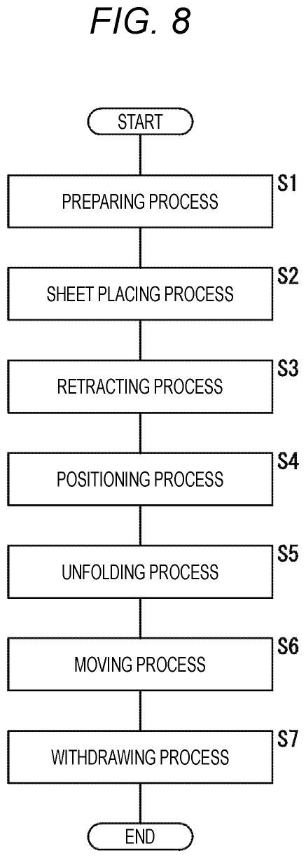

Next, a transfer method for transferring the medical sheet 300 to a treatment site of a living body will be described. Specifically, as illustrated in FIGS. 16 to 19, the transfer method for transferring the medical sheet 300 to the recipient site 402 of the heart 400 (the treatment site of the living body) during thoracoscopic surgery will be described. As illustrated in FIG. 8, the transfer method according to the present embodiment includes a preparing process, a sheet placing process, a retracting process, a positioning process, an unfolding process, a moving process, and a withdrawing process.

First, in the preparing process (step S1 in FIG. 8), the transfer instrument 10 according to the present embodiment described above is prepared. The following description will be given on the assumption that a state as illustrated in FIG. 1 is an initial state of the transfer instrument 10. In the initial state, the first and second shafts 24 and 48 are moved in the distal end direction (the direction of arrow X1) relative to the outer cylinder 22 so as to be at a protruding position (second position) where the first support portion 26 and the second support portion 50 protrude from the protection portion 78 (protrusion 782) of the outer cylinder 22 in the distal end direction. The first and second support portions 26 and 50 are each unfolded when moving out of the outer cylinder 22 in the distal end direction, and the second support portion 50 is positioned on the first support surface 261 of the first support portion 26. That is, the second support portion 50 is positioned at a retracted position where the second support portion 50 is placed on the first support surface 261 of the first support portion 26. At this time, the proximal portion of the carrier holding portion 54 is in the first lumen 28 of the first shaft 24.

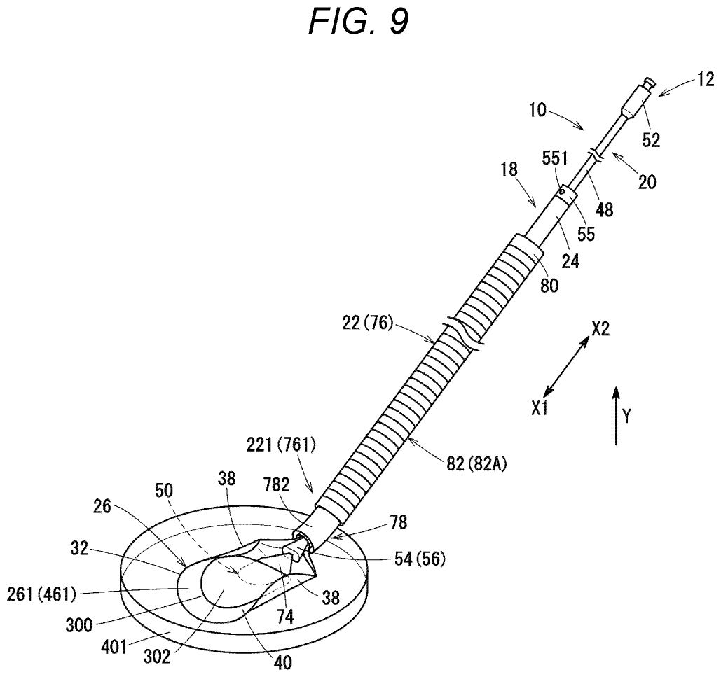

Next, in the sheet placing process (step S2 in FIG. 8), as illustrated in FIG. 9, the medical sheet 300 placed on a Petri dish 401 is placed on the second support surface 74. Note that, as illustrated in FIG. 10, the medical sheet 300 sticks out from the second support portion 50 with the medical sheet 300 placed on the second support surface 74. The first support surface 261 supports an overhanging portion 302 of the medical sheet 300 that sticks out from the second support portion 50. The pair of second protrusions 40 prevent the medical sheet 300 from moving (becoming misaligned) in the width direction (W direction) of the intermediate support portion 36 with the medical sheet 300 placed on the second support surface 74.

Thereafter, the retracting process (step S3 in FIG. 8) of retracting the medical sheet 300 into the outer cylinder 22 is performed. The medical sheet 300 is brought into a retraction position (first position) where the medical sheet 300 is retracted in the outer cylinder 22 together with the first support portion 26 and the second support portion 50. Specifically, the first shaft 24 of the first carrier member 18 and the second shaft 48 of the second carrier member 20 are moved together in the proximal end direction (the direction of arrow X2) relative to the outer cylinder 22.

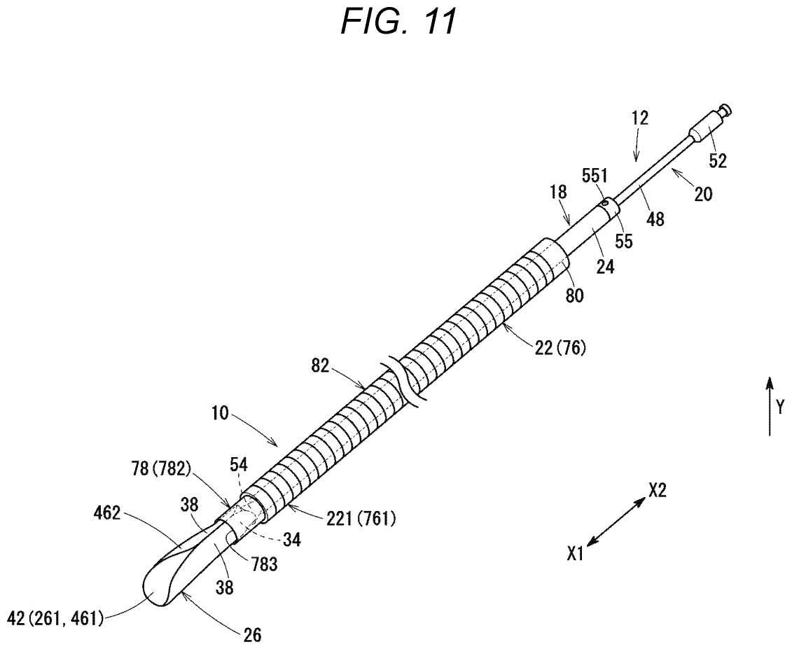

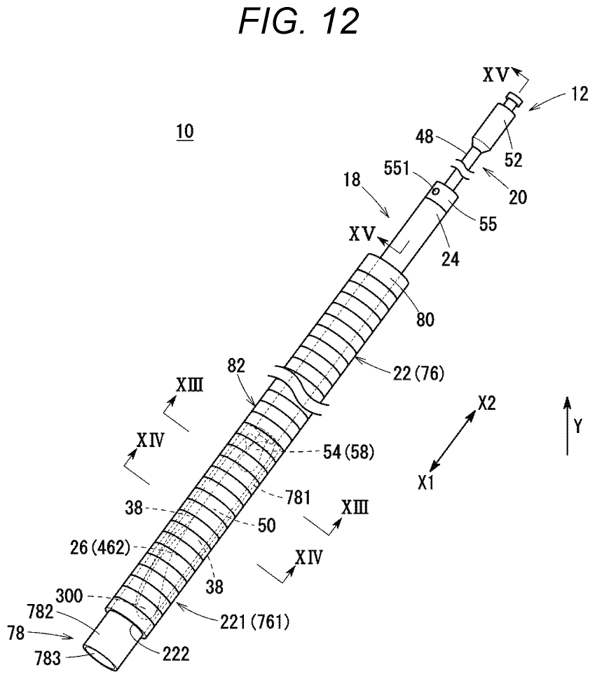

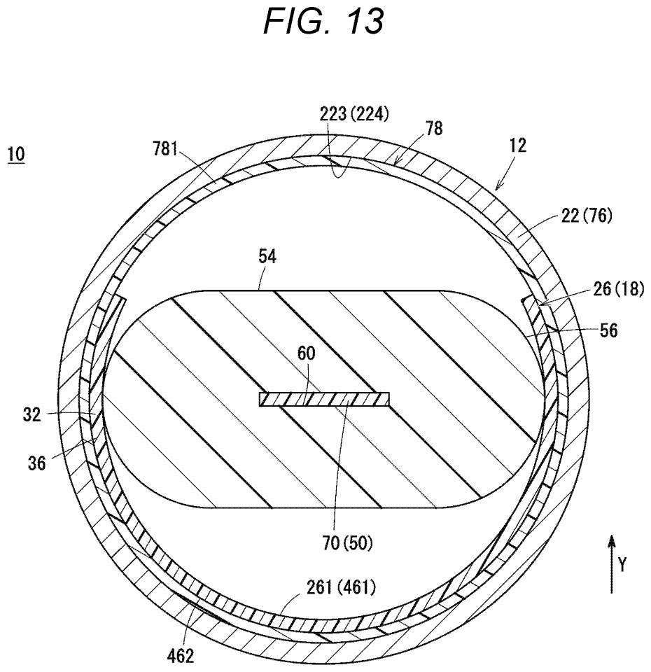

Then, as illustrated in FIG. 11, the proximal-end support portion 34 is pulled into the lumen 223 of the outer cylinder 22 in the proximal end direction through the through hole 783 of the protrusion 782. At this time, when both the tapered sides of the proximal-end support portion 34 come into contact with the distal end of the protrusion 782, a force acts on the proximal-end support portion 34 to cause the proximal-end support portion 34 to curl along the circumferential direction of the outer cylinder 22. Therefore, the proximal-end support portion 34 is smoothly pulled into the outer cylinder 22 through the protrusion 782 while curling. At this time, the first support portion 26 is prevented from coming into contact with the most distal end of the outer cylinder main body 76 (flexible portion 82) by coming into contact with the protrusion 782 of the protection portion 78. As illustrated in FIG. 12, the first support portion 26 is retracted into the outer cylinder 22 while curling in a conical shape such that the distal end side of the first support portion 26 becomes larger in diameter than the proximal-end support portion 34 (see FIG. 13).

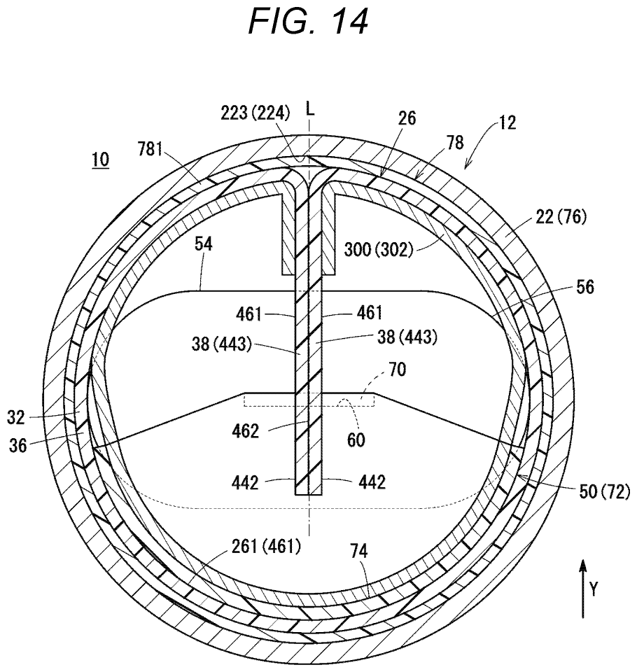

When the proximal-end support portion 34 becomes deformed, a force acts on the intermediate support portion 36 to cause the intermediate support portion 36 to curl along the circumferential direction of the outer cylinder 22, so that the intermediate support portion 36 is pulled into the outer cylinder 22 while curling. At this time, the intermediate support portion 36 becomes deformed into a cylindrical shape along the inner surface of the outer cylinder 22. As illustrated in FIG. 14, each of the pair of first protrusions 38 is curved such that its fixed end is rolled inward, and the front surface 461 of the first support portion 26 faces inward, and the back surface 462 of the first support portion 26 faces outward. The back surfaces 462 of intermediate portions 443 extending, in a convex shape, outward in the radial direction come into contact with each other on an imaginary line L extending in a direction orthogonal to a center axis of the outer cylinder 22. The intermediate portion 443 of one of the first protrusions 38 and the intermediate portion 443 of the other first protrusion 38 come into contact with each other and are retracted downward (toward the first support surface 261, the front surface 461).

Accordingly, the back surface 462 of the first support portion 26 is curved to come into close contact with the inner surface of the outer cylinder 22, each of the first protrusions 38 is further curved from the fixed end toward a free end 442 to fold back toward the center of the outer cylinder 22, and the pair of free ends 442 are positioned below the center axis of the outer cylinder 22. That is, the first support portion 26 is curved in a heart shape along the inner surface of the outer cylinder 22.

The heart shape refers to a roughly round shape including two convex shapes: a convex shape on one side and a convex shape on the other side. In a case where the heart shape is formed in the lumen 223 of the tubular body (outer cylinder 22), the two convex shapes protruding toward the opposite sides along the inner surface of the tubular body come close to each other so as to bring their respective peripheral surfaces into partial contact with each other, so that the entire contour becomes a roughly round shape along the inner surface of the tubular body (refer to the shape of the first support portion 26 in FIG. 14).

Along with the curved deformation of the first support portion 26, the second support portion 50 similarly undergoes curved deformation along the first support portion 26 inside the first support portion 26 (on the front surface 461 side). Along with the curved deformation of the first support portion 26 and the second support portion 50, the medical sheet 300 is deformed into a shape corresponding to the shape of the first support body 32 and the shape of the second support body 72, and the medical sheet 300 is retracted into the outer cylinder 22 accordingly. As illustrated in FIG. 12, when the first support portion 26 is entirely inserted into the outer cylinder 22, the retracting process is complete. At this time, as illustrated in FIG. 15, the stopper 481 provided on the second shaft 48 is caught in the valve 55 of the first shaft 24. The second shaft 48 is prevented from moving in the proximal end direction (direction of arrow X2) relative to the first shaft 24. That is, the second shaft 48 cannot be pulled in the proximal end direction from the first shaft 24.

In the retracted state where the first support portion 26, the second support portion 50, and the medical sheet 300 are retracted in the outer cylinder 22, as illustrated in FIG. 14, the pair of first protrusions 38 are located adjacent to the distal end (in the direction of arrow X1) relative to the pressing surface 58 of the pressure-application portion 56 with the back surfaces 462 in contact with each other (see FIG. 12).

Subsequently, in the positioning process (step S4 in FIG. 8), as illustrated in FIG. 16, a distal portion 221 of the outer cylinder 22 undergoes curved deformation by the flexible portion 82, and then the transfer instrument 10 is inserted into a chest cavity 410 through an incision 409 in a chest 408. At this time, the distal end of the transfer instrument 10 is positioned near the recipient site 402 of the heart 400. Note that before the transfer instrument 10 is inserted into the chest cavity 410, a liquid supply instrument may be connected to a connection port of the hub 52 to introduce a liquid (for example, a saline solution). Note that the transfer instrument 10 is not limited to the configuration where after the outer cylinder 22 undergoes curved deformation, the transfer instrument 10 is inserted into the chest cavity 410. For example, after the outer cylinder 22 is inserted into the chest cavity 410 in a linear state without undergoing curved deformation, the distal portion 221 of the outer cylinder 22 may undergo curved deformation by gripping the outer cylinder 22 (flexible portion 82) in the chest cavity 410 with forceps or the like.

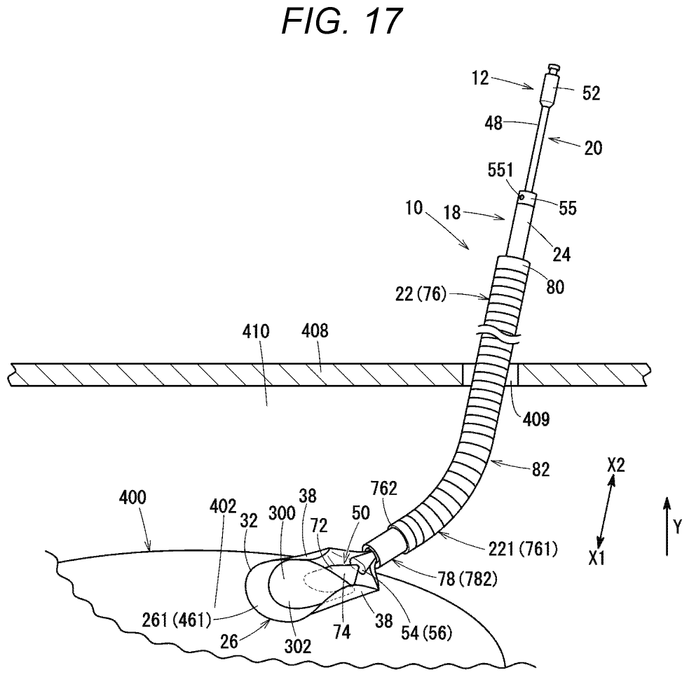

Subsequently, in the unfolding process (step S5 in FIG. 8), as illustrated in FIG. 17, the first support portion 26, the second support portion 50, and the medical sheet 300 are unfolded. Specifically, in the unfolding process, with the first shaft 24 gripped, the first shaft 24 is moved in the distal end direction (the direction of arrow X1) relative to the outer cylinder 22. Accordingly, the valve 55 of the first shaft 24 causes the second shaft 48 to move together with the first shaft 24 in the distal end direction (the direction of arrow X1). Then, the first support portion 26 that has moved out of the distal-end opening 222 of the outer cylinder 22 returns to the original shape due to its restoring force. At the second position where the first support portion 26 is unfolded, the second support portion 50 and the medical sheet 300 unroll flat.

In the unfolding process, of the second carrier member 20, the second support surface 74 on which the medical sheet 300 is placed is entirely positioned on the first support surface 261. At this time, the medical sheet 300 is supported by the first support surface 261 and the second support surface 74. It is therefore possible to prevent the overhanging portion 302 of the medical sheet 300 from becoming crinkled before the medical sheet 300 is transferred to the recipient site 402 of the heart 400.

Next, in the moving process (step S6 in FIG. 8), as illustrated in FIG. 18, the second carrier member 20 is moved in the distal end direction (the direction of arrow X1) relative to the first carrier member 18, so that the second support portion 50 on which the medical sheet 300 is placed is moved from the retracted position to an advanced position, and the second support portion 50 protrudes in the distal end direction (the direction of arrow X1) relative to the distal end of the first support portion 26. Specifically, in the moving process, the second shaft 48 is moved in the distal end direction relative to the first shaft 24.

Accordingly, the second support portion 50 moves in the distal end direction (the direction of arrow X1) relative to the first support portion 26. At this time, when the pressing surface 58 of the carrier holding portion 54 (pressure-application portion 56) presses the outer edge surface of the medical sheet 300 in the distal end direction, the medical sheet 300 is entirely positioned further than the first support portion 26 in the distal end direction. In this moving process, the medical sheet 300 is moved to above the recipient site 402 of the heart 400 to bring the overhanging portion 302 of the medical sheet 300 into contact with the recipient site 402.

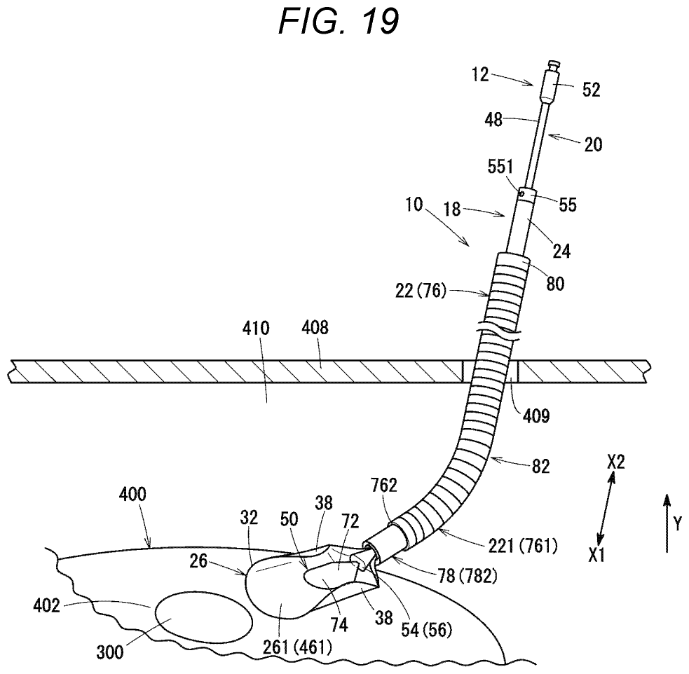

Subsequently, in the withdrawing process (step S7 in FIG. 8), as illustrated in FIG. 19, the second carrier member 20 is moved from the second position to the first position to withdraw the second support portion 50 from between the recipient site 402 and the medical sheet 300. Accordingly, the medical sheet 300 entirely comes into contact with the surface of the recipient site 402. This is the end of the transfer of the medical sheet 300 to the recipient site 402. Subsequently, the transfer instrument 10 is withdrawn from the chest 408 with the first support portion 26 and the second support portion 50 retracted in the outer cylinder 22.

The present embodiment has the following effects.

As illustrated in FIG. 1, the transfer instrument 10 has the flexible portion 82 that makes the distal portion 761 of the outer cylinder main body 76 flexible to undergo curved deformation and is capable of maintaining curved deformation. It is therefore possible to efficiently place the first support portion 26 of the first carrier member 18 at a desired position by effectively curving the distal portion 221 of the outer cylinder 22. The cylindrical protection portion 78 including an elastic material is provided at the distal portion 761 of the outer cylinder main body 76, and the inner peripheral surface 224 of the most distal end of the outer cylinder main body 76 is covered with the inner covering portion 781. Therefore, when the first support portion 26 is retracted into the lumen 223 of the outer cylinder 22, it is possible to effectively prevent the first support portion 26 from coming into contact with the inner peripheral surface 224 of the most distal end of the flexible portion 82.

As illustrated in FIG. 7A, when the first support portion 26 is retracted into the lumen 223 of the outer cylinder main body 76, at least a part of the first support portion 26 is disposed inside the flexible portion 82, which allows, as illustrated in FIG. 16, the distal portion 221 of the outer cylinder 22 to undergo curved deformation by the flexible portion 82 even with the first support portion 26 retracted.

As illustrated in FIG. 2, the protection portion 78 includes the protrusion 782 protruding in the distal end direction (the direction of arrow X1) from the most distal end 762 of the outer cylinder main body 76, which allows, as illustrated in FIG. 11, when the first support portion 26 is retracted into the outer cylinder 22, the first support portion 26 to be effectively guided to the lumen 223 of the outer cylinder 22 by the protrusion 782.

As illustrated in FIG. 7A, the inner covering portion 781 has a length longer than or equal to half of the first support portion 26 in the axial direction of the outer cylinder main body 76, which prevents, when the first support portion 26 is retracted into the outer cylinder 22, the outer cylinder 22 (outer cylinder main body 76) and the first support portion 26 from coming into direct contact with each other.

As illustrated in FIG. 2, the flexible portion 82 is a tubular body obtained by winding, in a spiral shape, the plate members 82A including a metal material. This makes the flexible portion 82 simple in configuration and low in cost.

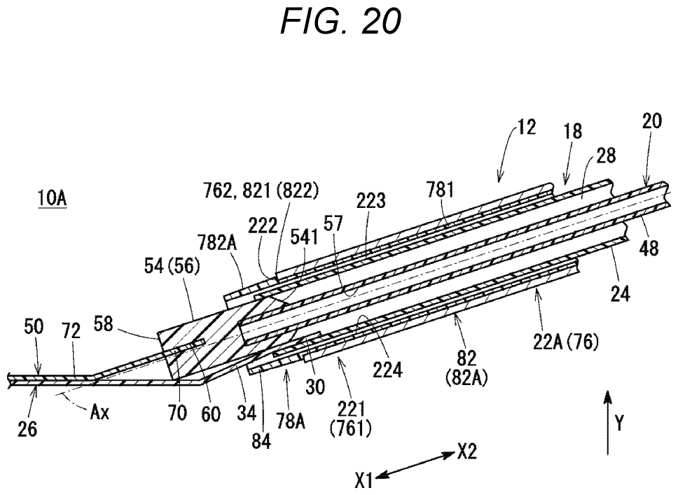

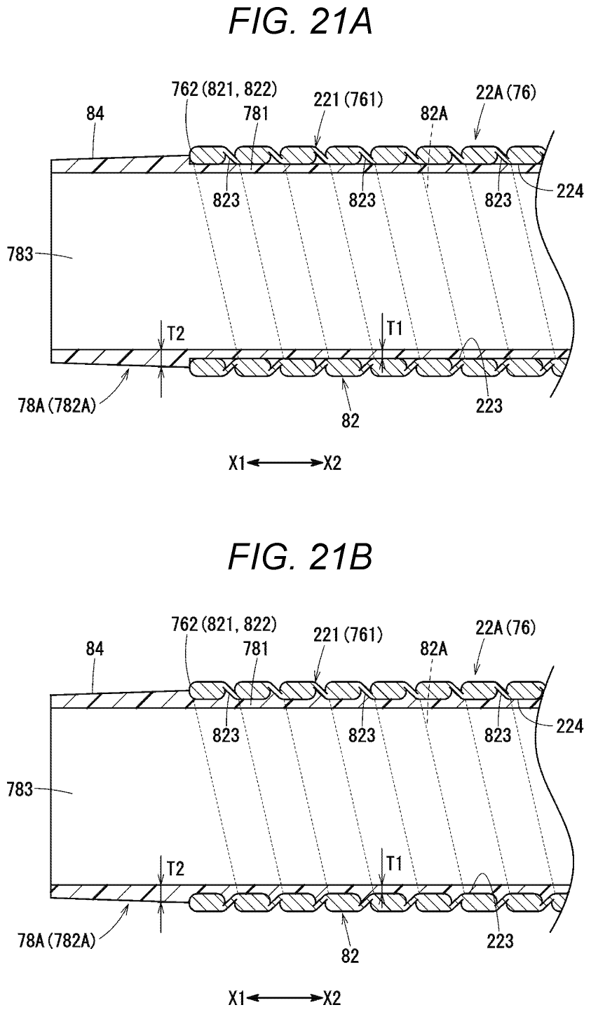

A transfer instrument 10A according to a first modification illustrated in FIG. 20 includes an outer cylinder 22A. The outer cylinder 22A includes a protection portion 78A. As illustrated in FIG. 21A, the protection portion 78A is formed by, for example, injection molding. The protection portion 78A includes the inner covering portion 781 and a protrusion 782A. The inner covering portion 781 covers the step portion 823 of the flexible portion 82. As illustrated in FIG. 21B, a part of the inner covering portion 781 may be configured to enter a space formed by the step portion 823. The part of the inner covering portion 781 can enter the space formed by the step portion 823 by, for example, fusion or insert molding. As illustrated in FIG. 21A, the inner peripheral surface 224 of the most distal end of the outer cylinder main body 76 is covered with the inner covering portion 781. A thickness T2 of the protrusion 782A is larger than a thickness T1 of the inner covering portion 781 in the radial direction of the outer cylinder 22A (T1<T2). A proximal end of the protrusion 782A covers a part of a most distal end surface 822 of the outer cylinder main body 76. An outer peripheral surface 84 of the protrusion 782A is formed in a tapered shape in the distal end direction. The inner covering portion 781 of the protection portion 78A is inserted into the lumen 223 from the distal-end opening 222 of the outer cylinder main body 76, and the inner covering portion 781 is fitted to the inner peripheral surface 224 of the outer cylinder main body 76.

The first modification has the following effects.

As illustrated in FIG. 21A, the protection portion 78A of the transfer instrument 10A is formed to make the protrusion 782A thicker than the inner covering portion 781 in the radial direction of the outer cylinder 22A, which allows an increase in rigidity of the protrusion 782A protruding from the most distal end 762 of the outer cylinder main body 76. This in turn allows, when the first support portion 26 is retracted into the lumen 223 of the outer cylinder 22A, the first support portion 26 to be effectively guided into the outer cylinder 22A by the protrusion 782A.

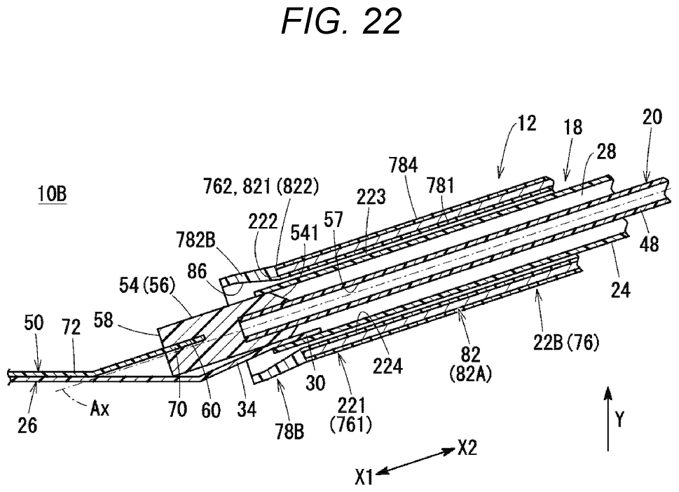

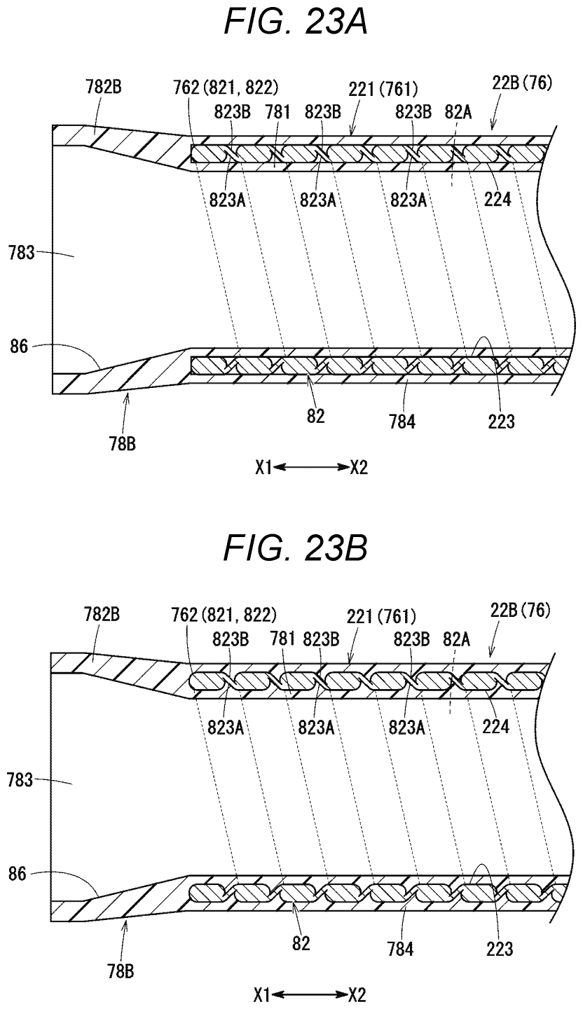

A transfer instrument 10B according to a second modification illustrated in FIG. 22 includes an outer cylinder 22B. The outer cylinder 22B includes a protection portion 78B. As illustrated in FIG. 23A, the protection portion 78B includes the inner covering portion 781, a protrusion 782B, and an outer covering portion 784. An inner peripheral surface of the protrusion 782B includes a reverse tapered portion 86 whose diameter increases toward an end in the distal end direction (the direction of arrow X1). The reverse tapered portion 86 has a maximum diameter at a distal end of the protrusion 782B. A proximal end of the reverse tapered portion 86 is almost identical in diameter to the lumen 223 of the outer cylinder 22B.

The outer covering portion 784 covers an outer peripheral surface of the flexible portion 82 and extends in the proximal end direction (the direction of arrow X2) from a proximal end of the protrusion 782B. The outer covering portion 784 covers the outer peripheral surface of the flexible portion 82 over the entire length of the flexible portion 82 in the axial direction of the outer cylinder main body 76. Note that the outer covering portion 784 is not limited to the configuration where the outer covering portion 784 covers all over the outer peripheral surface of the flexible portion 82 in the axial direction of the outer cylinder main body 76. The outer covering portion 784 may cover at least the distal portion 221 including the most distal end of the outer cylinder main body 76 (flexible portion 82).

The inner covering portion 781 and the outer covering portion 784 cover an inner step portion 823A and an outer step portion 823B of the flexible portion 82, respectively. Note that the inner covering portion 781 and the outer covering portion 784 are not limited to the configuration where the inner covering portion 781 covers the inner peripheral surface 224 of the flexible portion 82 with a space provided between the inner covering portion 781 and the inner step portion 823A, and the outer covering portion 784 covers the outer peripheral surface of the flexible portion 82 with a space provided between the outer covering portion 784 and the outer step portion 823B. For example, as illustrated in FIG. 23B, a part of the inner covering portion 781 may be configured to enter the space formed by the inner step portion 823A, and a part of the outer covering portion 784 may be configured to enter the space formed by the outer step portion 823B. In this configuration, when the flexible portion 82 undergoes curved deformation, the part of the inner covering portion 781 entering the inner step portion 823A may be pushed radially inward, and the part of the outer covering portion 784 entering the outer step portion 823B may be pushed radially outward.

When the first support portion 26 is retracted into the outer cylinder 22B from the state illustrated in FIG. 22 where the first support portion 26 is unfolded, the first support portion 26 is guided in the proximal end direction along the reverse tapered portion 86 of the protrusion 782B. The first support portion 26 is curved by the reverse tapered portion 86 so as to gradually reduce in diameter toward the outer cylinder 22B. The curved first support portion 26 is retracted into the lumen 223 of the outer cylinder 22B from the proximal end of the protrusion 782B (reverse tapered portion 86).

The second modification has the following effects.

The protection portion 78B of the transfer instrument 10B includes the outer covering portion 784 that covers the outer peripheral surface of the flexible portion 82, and the outer covering portion 784 extends in the proximal end direction (the direction of arrow X2) from the protrusion 782B, which allows, when the outer cylinder 22B is inserted into the living body, the outer covering portion 784 to effectively prevent the flexible portion 82 from coming into contact with the living body.

Since the inner peripheral surface of the protrusion 782B includes the reverse tapered portion 86 whose diameter increases toward the end in the distal end direction, when the first support portion 26 is retracted into the lumen 223 of the outer cylinder 22B, the reverse tapered portion 86 can guide the first support portion 26 in the proximal end direction to effectively retract the first support portion 26 into the outer cylinder 22B.

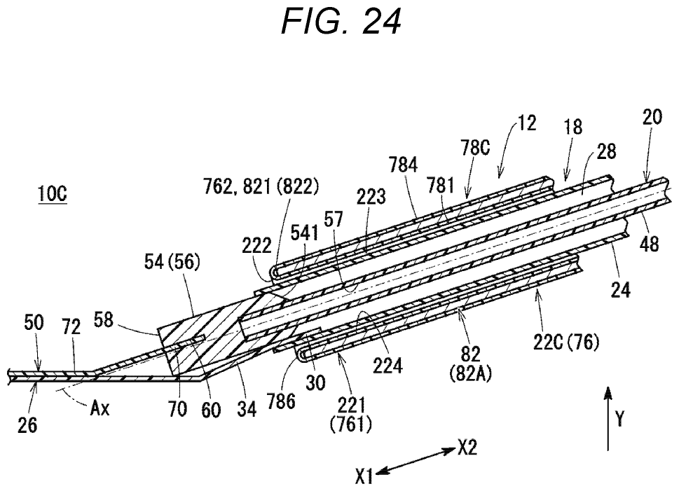

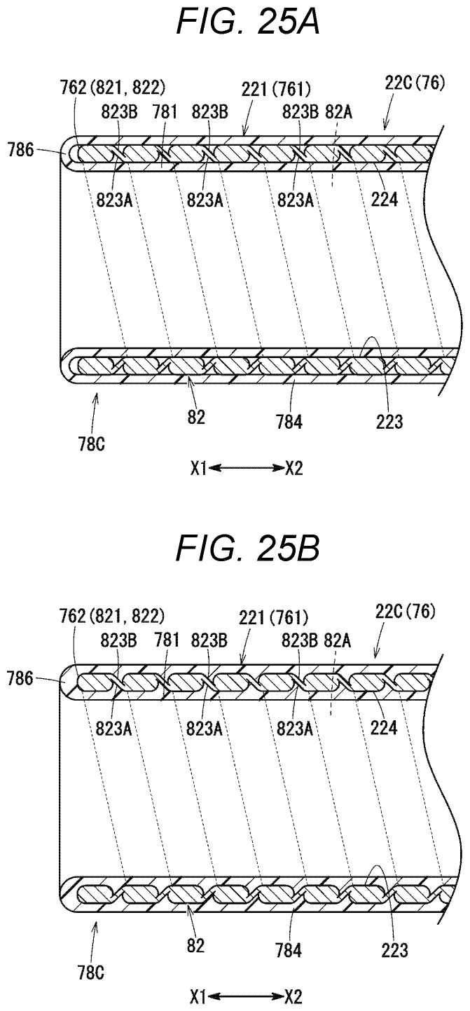

A transfer instrument 10C according to a third modification illustrated in FIG. 24 includes an outer cylinder 22C. The outer cylinder 22C includes a protection portion 78C. As illustrated in FIG. 25A, the protection portion 78C includes the inner covering portion 781, the outer covering portion 784, and a distal-end covering portion 786. The distal-end covering portion 786 covers the most distal end 762 of the outer cylinder main body 76. A distal end of the outer covering portion 784 and a distal end of the inner covering portion 781 are connected to each other by the distal-end covering portion 786. The distal-end covering portion 786 has a curved shape that is convex in a direction (distal end direction) away from the most distal end 762 of the outer cylinder main body 76. Note that the distal-end covering portion 786 is not limited to the configuration where the distal-end covering portion 786 is formed in a curved shape. For example, the distal-end covering portion 786 may be a flat surface along the most distal end surface 822 of the outer cylinder main body 76.

The inner covering portion 781 and the outer covering portion 784 cover an inner step portion 823A and an outer step portion 823B of the flexible portion 82, respectively. Note that the inner covering portion 781 and the outer covering portion 784 are not limited to the configuration where the inner covering portion 781 covers the inner peripheral surface 224 of the flexible portion 82 with a space provided between the inner covering portion 781 and the inner step portion 823A, and the outer covering portion 784 covers the outer peripheral surface of the flexible portion 82 with a space provided between the outer covering portion 784 and the outer step portion 823B. For example, as illustrated in FIG. 25B, a part of the inner covering portion 781 may be configured to enter the space formed by the inner step portion 823A, and a part of the outer covering portion 784 may be configured to enter the space formed by the outer step portion 823B. In this configuration, when the flexible portion 82 undergoes curved deformation, the part of the inner covering portion 781 entering the inner step portion 823A may be pushed radially inward, and the part of the outer covering portion 784 entering the outer step portion 823B may be pushed radially outward.

The third modification has the following effects.

As illustrated in FIG. 25A, the protection portion 78C of the transfer instrument 10C includes the distal-end covering portion 786 that covers the most distal end 762 of the outer cylinder main body 76. It is therefore possible to prevent, by covering all over the most distal end 762 of the outer cylinder main body 76 with the distal-end covering portion 786, the first support portion 26 and the distal portion 761 of the outer cylinder main body 76 from coming into contact with each other more effectively when the first support portion 26 is retracted into the outer cylinder 22C.

The protection portion 78C includes the outer covering portion 784 that covers the outer peripheral surface of the flexible portion 82, and the distal end of the outer covering portion 784 and the distal end of the inner covering portion 781 are connected to each other by the distal-end covering portion 786, which allows the outer covering portion 784, the inner covering portion 781, and the distal-end covering portion 786 to effectively cover the distal portion 221 of the outer cylinder 22C.

The detailed description above describes embodiments of a transfer instrument. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A transfer instrument configured to transfer a medical sheet to a treatment site of a living body, the transfer instrument comprising:
an outer cylinder including a distal-end opening;
a first carrier member including a first shaft and a support portion having a sheet shape, the first shaft extending in an axial direction of the outer cylinder and being disposed inside the outer cylinder so as to be movable along the axial direction, the support portion being disposed at a distal portion of the first shaft and including a support surface configured to hold the medical sheet;
a second carrier member including a second shaft and a pressure-application portion, the second shaft extending along the first shaft and being provided so as to be movable along the first shaft, the pressure-application portion being provided at a distal portion of the second shaft to press the medical sheet in a distal end direction;
wherein the outer cylinder includes:
an outer cylinder main body having a lumen communicating with the distal-end opening; and
a protection portion having a tubular shape, the protection portion including an elastic material and being provided at a distal portion of the outer cylinder main body;
at least the distal portion of the outer cylinder main body includes a flexible portion that is flexible to undergo curved deformation and is capable of maintaining curved deformation;
the flexible portion is provided at least at a distal portion of the outer cylinder;
the protection portion includes an inner covering portion that covers at least an inner peripheral surface of a most distal end of the outer cylinder main body and opens in the distal end direction to communicate with the lumen of the outer cylinder main body;
in a state where the first and second shafts are moved in the distal end direction relative to the outer cylinder so as to cause the support portion to protrude from the distal-end opening of the outer cylinder, the support portion is unfolded by being exposed from the outer cylinder in the distal end direction; and
in a state where the support portion is unfolded, when the first and second shafts are moved in a proximal end direction relative to the outer cylinder, the support portion is retracted into the lumen of the outer cylinder through the inner covering portion.

2. The transfer instrument according to claim 1, wherein
when the support portion is retracted into the lumen of the outer cylinder main body, at least a part of the support portion is disposed inside the flexible portion.

3. The transfer instrument according to claim 1, wherein
the protection portion includes a protrusion protruding from the most distal end of the outer cylinder main body in the distal end direction.

4. The transfer instrument according to claim 3, wherein
the protection portion includes an outer covering portion that covers an outer peripheral surface of the flexible portion, and
the outer covering portion extends from the protrusion in the proximal end direction.

5. The transfer instrument according to claim 4, wherein
the outer covering portion covers the outer peripheral surface of the flexible portion over an entire length of the flexible portion in the axial direction of the outer cylinder main body.

6. The transfer instrument according to claim 1, wherein
the inner covering portion has a length of half or more of the support portion in the axial direction of the outer cylinder main body.

7. The transfer instrument according to claim 3, wherein
the protrusion is thicker than the inner covering portion in a radial direction of the outer cylinder.

8. The transfer instrument according to claim 3, wherein
an inner peripheral surface of the protrusion includes a reverse tapered portion whose diameter increases toward an end in the distal end direction.

9. The transfer instrument according to claim 8, wherein
the protection portion includes an outer covering portion that covers an outer peripheral surface of the flexible portion and extends from the protrusion in the proximal end direction.

10. The transfer instrument according to claim 1, wherein
the protection portion includes a distal-end covering portion that covers the most distal end of the outer cylinder main body.

11. The transfer instrument according to claim 10, wherein
the protection portion includes an outer covering portion that covers an outer peripheral surface of the flexible portion, and
a distal end of the outer covering portion and a distal end of the inner covering portion are connected to each other by the distal-end covering portion.

12. The transfer instrument according to claim 1, wherein
the flexible portion is a tubular body obtained by winding, in a spiral shape, a plate member including a metal material.

13. A transfer instrument configured to transfer a medical sheet to a treatment site of a living body, the transfer instrument comprising:
an outer cylinder including a distal-end opening;
a first carrier member including a first shaft and a support portion having a sheet shape, the first shaft extending in an axial direction of the outer cylinder and being disposed inside the outer cylinder so as to be movable along the axial direction, the support portion being disposed at a distal portion of the first shaft and including a support surface configured to hold the medical sheet;
a second carrier member including a second shaft and a pressure-application portion, the second shaft extending along the first shaft and being provided so as to be movable along the first shaft, the pressure-application portion being provided at a distal portion of the second shaft to press the medical sheet in a distal end direction;
the outer cylinder including an outer cylinder main body having a lumen communicating with the distal-end opening and a protection portion having a tubular shape, the protection portion including an elastic material and being provided at a distal portion of the outer cylinder main body;
at least the distal portion of the outer cylinder main body includes a flexible portion that is flexible to undergo curved deformation and is configured to maintain a curved deformation;
the flexible portion is provided at least at a distal portion of the outer cylinder;
the protection portion includes an inner covering portion that covers at least an inner peripheral surface of a most distal end of the outer cylinder main body and opens in the distal end direction to communicate with the lumen of the outer cylinder main body; and
wherein the protection portion includes a protrusion protruding from the most distal end of the outer cylinder main body in the distal end direction, and an outer covering portion that covers an outer peripheral surface of the flexible portion, the outer covering portion extending from the protrusion in the proximal end direction, and the outer covering portion covers the outer peripheral surface of the flexible portion over an entire length of the flexible portion in the axial direction of the outer cylinder main body.

14. The transfer instrument according to claim 13, wherein
in a state where the first and second shafts are moved in the distal end direction relative to the outer cylinder so as to cause the support portion to protrude from the distal-end opening of the outer cylinder, the support portion is unfolded by being exposed from the outer cylinder in the distal end direction, and
in a state where the support portion is unfolded, when the first and second shafts are moved in a proximal end direction relative to the outer cylinder, the support portion is retracted into the lumen of the outer cylinder through the inner covering portion.

15. The transfer instrument according to claim 14, wherein
when the support portion is retracted into the lumen of the outer cylinder main body, at least a part of the support portion is disposed inside the flexible portion.

16. The transfer instrument according to claim 14, wherein
the inner covering portion has a length of half or more of the support portion in the axial direction of the outer cylinder main body.

17. The transfer instrument according to claim 14, wherein
the protrusion is thicker than the inner covering portion in a radial direction of the outer cylinder; and
an inner peripheral surface of the protrusion includes a reverse tapered portion whose diameter increases toward an end in the distal end direction.

18. A method for transferring a medical sheet to a treatment site of a living body, the method comprising:
placing a medical sheet on a support surface of a medical instrument, the medical instrument including an outer cylinder including a distal-end opening, a first carrier member including a first shaft and a support portion having a sheet shape, the first shaft extending in an axial direction of the outer cylinder and being disposed inside the outer cylinder so as to be movable along the axial direction, the support portion being disposed at a distal portion of the first shaft and the support surface; and
pressing the medical sheet in a distal end direction with a second carrier member, the second carrier member including a second shaft and a pressure-application portion, the second shaft extending along the first shaft and being provided so as to be movable along the first shaft, the pressure-application portion being provided at a distal portion of the second shaft, and wherein the outer cylinder includes an outer cylinder main body having a lumen communicating with the distal-end opening and a protection portion having a tubular shape, the protection portion including an elastic material and being provided at a distal portion of the outer cylinder main body.

19. The method according to claim 18, wherein at least the distal portion of the outer cylinder main body includes a flexible portion that is flexible to undergo curved deformation and is capable of maintaining curved deformation, the flexible portion is provided at least at a distal portion of the outer cylinder, the protection portion includes an inner covering portion that covers at least an inner peripheral surface of a most distal end of the outer cylinder main body and opens in the distal end direction to communicate with the lumen of the outer cylinder main body, the method comprises:
moving the first shaft and the second shaft in the distal end direction relative to the outer cylinder so as to cause the support portion to protrude from the distal-end opening of the outer cylinder; and
retracting the support portion into the lumen of the outer cylinder through the inner covering portion to withdraw the second support portion from between the treatment site and the medical sheet.

20. The method according to claim 18, further comprising:
bringing the medical sheet into contact with the treatment site.
